(19) United States
(12) Patent Application Publication
(10) Pub. No.: US 2005/0096693 A1
(43) Pub. Date: May 5, 2005

(54) Apparatus and technique for releasing congested prostate fluid

(76) Inventor: Plato Rosinke, New York, NY (US)

Correspondence Address: PLATO ROSINKE, 200 E 36 STREET, NEW YORK, NY, 10016, US

(21) Appl. No.: 10/700,267

(22) Filed: Nov. 4, 2003

(51) Int. Cl. A61B 17/00

(52) U.S. Cl. 606/204

(57) ABSTRACT

An apparatus for releasing congested prostate fluid including a section of animal horn having a conical point on at least one end of a size suitable for applying pressure against the abdominal wall and perineum region.

### CROSS REFERENCE TO RELATED APPLICATIONS

Not applicable.

### STATEMENT REGARDING RED SPONSORED R & D

There has been no Fed sponsored R & D with respect to this application.

### REFERENCE TO SEQUENCE LISTING, A TABLE, OR A COMPUTER PROGRAM LISTING APPENDIX

Not applicable.

### BRIEF SUMMARY OF THE INVENTION

The present invention relates to an apparatus for the treatment of prostatitis. More particularly, the present invention relates to a device and specific methodology for massaging the prostate gland.

The present invention is an apparatus for do-it-yourself release of congested prostate fluid. This apparatus allows an individual to induce deep and steady pressure against the abdominal wall thus making contact with the prostate gland and surrounding tissues so as to express the fluid from the congested prostate.

This apparatus also allows an individual to reflexively stimulating the prostate via the perineum region so as to express the fluid from the congested prostate.

### BACKGROUND OF THE INVENTION

One treatment for non-bacterial disorder of prostate such as chronic prostatitis and a congested prostate is the prostate massage. Some urologists believe that the most effective treatment for such prostatitis is for the doctor to massage the prostate at regular intervals. In any event, the regular and repeated massaging of the prostate can often require frequent visits to the doctor's office. This causes the patient to incur a considerable expense and an inconvenience. As such, a need has developed for allowing an individual to carry out his own prostatic massage.

In the past, some patents have issued relating to rectal devices U.S. Pat. No. 4,542,753, issued on Sep. 23, 1985 to Brenman et Describes an apparatus and method for stimulating penile erectile tissue. In this invention, a body is provided which may be inserted into the rectum of a user. The body is shaped so as to closely conform to the topological configuration of the rectum within the anal area to a site adjacent to the prostate gland. Electrical circuitry for generating a neurally stimulating electrical signal is located within the body. Electrodes, placed at particular locations on the surface of the body, apply the signal to the user. At least one of the electrodes closely contacts the prostate gland when the body member is operatively disposed, at a region or spot on the prostate gland previously determined to be sensitive to electrical stimulation.

U.S. Pat. No. 5,404,881, issued on Apr. 11, 1995, to Cathaud et al. describes a trans-rectal probe. This trans-rectal probe includes a probe body made of a flexible self-supporting polymer material whose degree of flexibility is designed to enable it to comply with the shape therein. The invention makes it possible to achieve safe and reliable positioning of an instrument for detection or therapeutic treatment level with the organ to be observed or treated. In particular. This device is designed for treatment of the prostate.

U.S. Pat. No. 2,478,786, issued on Aug. 9, 1949 to H. M. Smallen describes a prostate gland massaging implement. This implement includes a lever having an interior handle which constitutes a power arm to extend down in front of the abdomen and a substantially horizontal portion extending under the groin and offset laterally to avoid the genital organs. The implement has an upwardly and forwardly bent posterior portion which forms the work arm. This work arm extends into the rectal passage to bear against the frontal wall thereof adjacent the prostate gland. The bent portion between the horizontal and the posterior portions serves as a fulcrum point against the front wall of the rectal opening when the implement is subject to pivotal movement around this point.

It is an object of the present invention to provide an apparatus which enables persons to carry out self-massages of the prostate without inserting anything into the anus or the rectum so as to express the fluid from the congested prostate.

It is an object of the present invention to provide an apparatus which provides pressure on the prostate gland via deep massage of the abdominal wall as well as reflexively stimulating the prostate via the perineum region.

It is a further object of the present invention to provide an apparatus for a prostate massage which is safe, easy to use, and inexpensive.

### BRIEF DESCRIPTION OF THE DRAWINGS

FIG. 1 is an angled view of the apparatus of the present invention.

FIG. 2 is a view of the apparatus of FIG. 1 at an alternative angle.

### DETAILED DESCRIPTION

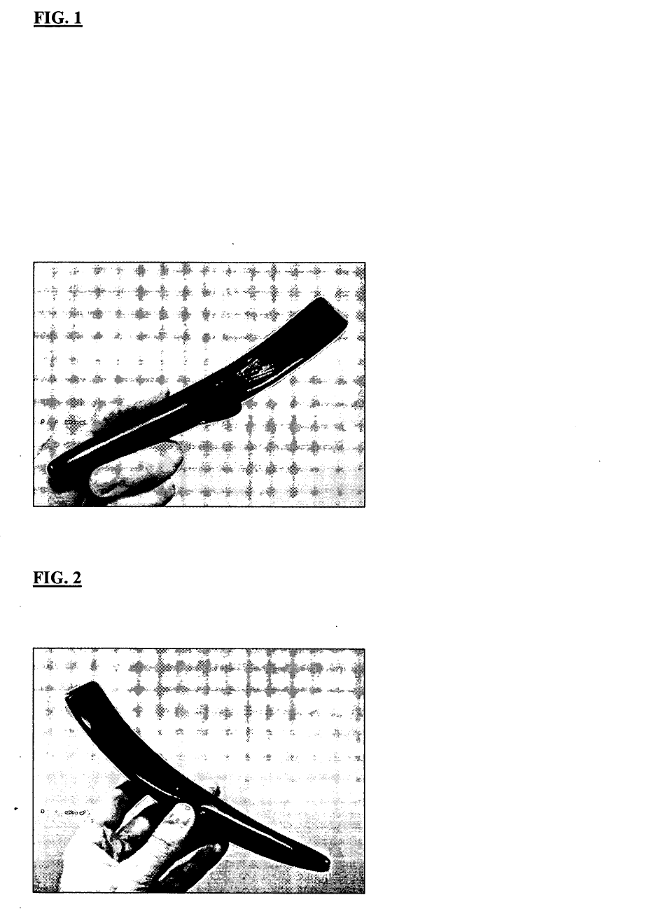

FIG. 1 shows the apparatus of the present invention for massaging the prostate via the abdominal wall and perineum region to release congested prostate fluid. The material that the apparatus is made from is animal horn, and is shaped and laquered before use.

FIG. 2 more clearly details the bevelling that occurs at one end and the conical point at the other.

1. An apparatus for releasing congested prostate fluid comprising:
a length of animal horn that tapers into a smooth, conical point on at least one end that has a diameter of between ¼″ and ½″ such is suitable for applying pressure to the abdominal wall and perineum region.

2. The apparatus of claim 1, said animal horn means comprising:
a section of animal horn having a generally L-shaped or C-shaped configuration, said configuration having a radius of curvature that allows for various angles of approach against the abdominal wall and perineum.
